(19) United States
(12) Patent Application Publication
(10) Pub. No.: US 2009/0263828 A1
(43) Pub. Date: Oct. 22, 2009

(54) Novel assay for precursor T-cells having high proliferative capacity (PHPC-asay)

(76) Inventors: Franco Lori, Bethesda, MD (US); Sandra A. Calarota, Pavia (IT); Julianna Lisziewicz, Bethesda, MD (US)

Correspondence Address: THE LAW OFFICES OF VALERIE E. LOOPER, 11726 LIGHTFALL COURT, COLUMBIA, MD, 21044, US

(21) Appl. No.: 12/386,333

(22) Filed: Apr. 16, 2009

Related U.S. Application Data
(60) Provisional application No. 61/124,376, filed on Apr. 16, 2008.

(51) Int. Cl. G01N 33/53

(52) U.S. Cl. 435/7.2

(57) ABSTRACT

Diagnostic assay of antigen-specific T-cell precursors to determine the immunological status of patients suffering in chronic infectious diseases and to anticipate the disease progression and the response to therapy

### RELATED APPLICATION

This application is a continuation-in-part of U.S. provisional application No. 61/124,376 filed 16 Apr. 2008, which is incorporated herein by reference as if set forth in full.

### FIELD OF THE INVENTION

This invention relates to a novel analytical method, which can detect precursors to T-cells that have high proliferative capacity in patients suffering of chronic infectious, neoplastic and allergic diseases. This method can be utilized to guide a patient's disease management and to predict long-term success or failure of human drug treatments as well as prophylactic and/or therapeutic vaccines.

The invention relates to a novel test to analyze and quantify the antigen-specific T-cell Precursors with High Proliferative Capacity (PHPC) as an immune diagnostic tool to evaluate the immunological status of patients suffering in chronic infectious, neoplastic and allergic diseases where antigen-specific cytotoxic T-cell precursors correlate with disease progression. The utility of this assay is demonstrated in individuals who are chronically infected with HIV: the quantity of HIV-specific T-cell precursors with high proliferative capacity (HIV-PHPC counts) correlated with the patients' immunological status better than the currently accepted CD4 counts. In addition, HIV-PHPC counts correlated with the disease progression of untreated patients in a longitudinal study. The preferred detection method is to use selected antigen-specific peptides in a long-term cultured ELISPOT assay. This test has the advantage of detecting the presence of a class of cells that is capable of handling long-term immunological challenge. This test can be used to determine the ability of prophylactic and therapeutic approaches to produce changes in the immune system status, or competence, of individuals with the above listed chronic diseases, and to predict long-term success or failure of treatments, including antiretroviral therapy and immune therapies based on immune modulation and targeted immune amplification, and also to guide a patient's disease management.

### BACKGROUND OF THE INVENTION

Several lines of evidence suggest that antigen-specific T-cell responses play a critical role in controlling chronic infections and cancer in humans. See Borrow P, Lewicki H, Hahn B H, et al. Virus-specific CD8+ cytotoxic T-lymphocyte activity associated with control of viremia in primary human immunodeficiency virus type 1 infection. J Virol 1994; 68:6103-10; Pape G R, Gerlach T J, Diepolder H M, et al. Role of the specific T-cell response for clearance and control of hepatitis C virus. J Viral Hepat 1999; 6 Suppl 1:36-40; Maini M K, Bertoletti A. How can the cellular immune response control hepatitis B virus replication?; Knutson K L, Disis M L. Augmenting T helper cell immunity in cancer. Curr Drug Targets Immune Endocr Metabol Disord 2005; 5:365-71.

Although several lines of evidence suggest that antigen-specific CD4+ and CD8+ T-cell responses play a critical role in controlling HIV infection in humans and SIV in the macaque model, the precise immune correlates of HIV control remain to be identified.

Recent studies in nonhuman primate models for AIDS indicate that the preservation of vaccine-induced, antigen-specific central memory CD4+ T-cells is essential for better outcome and survival following pathogenic SIV challenge, and also that vaccine-induced, virus-specific central memory CD8+ T-cells inversely correlate with virus levels in challenged macaques. However, the association of antigen-specific central memory T-cells with viral load level and CD4 counts in HIV infection have not been established. A distinct feature of central memory T-cells is their ability to proliferate after antigen re-exposure. After proliferation, central memory T-cells acquire effector functions, such as interferon-gamma (IFN-γ) secretion. Once the central memory T-cells become effector cells, they have a reduced ability to proliferate although they retain the ability to secrete IFN-γ. Therefore, an assay that can quantify antigen-specific circulating T-cell precursors with high proliferative capacity (PHPC) instead of just the ability to secrete IFN-γ, might be used to evaluate correlates of viral control and positive immunologic outcome in natural HIV infection. Additionally, they may provide an effective tool to predict treatment outcome.

The IFN-γ enzyme-linked immunospot (ELISPOT) assay is currently used to detect HIV-specific T-cell responses in humans. This assay quantifies T-cells secreting IFN-γ within 18-24 hours of antigen stimulation, that is, short-lived effector T-cells. There are similar assays that detect similar immune responses after short antigenic stimulation using flow cytometric detection of IFN-γ or other cytokines.

The ELISPOT assay does not correlate with a patient's ability to suppress HIV viral replication or with positive immunological outcome. PBMC (cells that are a blood product derivative) from 32 chronically untreated HIV-infected individuals were first evaluated for response to antigenic peptide pools, that is, a mixture of peptides known to be antigenic fragments of proteins, representing the complete HIV-1 Gag, Nef and Rev proteins, by using the standard IFN-γ ELISPOT assay (ELISPOT). This approach did not yield any meaningful correlation between immune responses and HIV viral suppression.

Therefore, we modified the standard ELISPOT assay. In the new assay, cells are first cultured with the antigen for 12 days in order to allow precursor T-cells to proliferate in response to antigen and to acquire effector function. In the meantime, the antigen-stimulated effector T-cells from the original sample should undergo apoptosis, or normal cell death. Therefore, this new assay quantifies antigen-specific T-cell precursors with high proliferative capacity (PHPC assay), without interference from the existing set of effector cells, and provides a new way of measuring long-lasting memory T-cells. In the prior art it has been reported that similar antigen-specific T-cells measured with cultured ELISPOT correlated with protection against malaria [See Reece W H, Pinder M, Gothard P K, et al. A CD4(+) T-cell immune response to a conserved epitope in the circumsporozoite protein correlates with protection from natural Plasmodium falciparum infection and disease. Nat Med 2004; 10:406-10; Keating S M, Bejon P, Berthoud T, et al. Durable human memory T cells quantifiable by cultured enzyme-linked immunospot assays are induced by heterologous prime boost immunization and correlate with protection against malaria. J Immunol 2005; 175:5675-80; Webster D P, Dunachie S, Vuola J M, et al. Enhanced T cell-mediated protection against malaria in human challenges by using the recombinant poxviruses FP9 and modified vaccinia virus Ankara. Proc Natl Acad Sci U S A 2005; 102:4836-41.] and with suppression of viral rebound in chronic hepatitis B carriers [See Yang S H, Lee C G, Park S H, et al. Correlation of antiviral T-cell responses with suppression of viral rebound in chronic hepatitis B carriers: a proof-of-concept study. Gene Ther 2006; 13:1110-7.]. However, these findings were (1) specific to the diseases of malaria and hepatitis B, not for HIV or other chronic diseases; (2) we do not know the details of these tests, and consequently (3) we cannot know whether the same T-cell precursor population that we detect with our assay can be detected with the assay described in prior art. Moreover, here we have standardized and optimized the test to be used as diagnostic tool. However, evaluation of T-cell responses by PHPC assay in HIV-infected individuals has not been described previously.

### BRIEF DESCRIPTION OF THE DRAWINGS

FIG. 1. HIV-1-specific T-cell responses determined by IFN-γ ELISPOT (ELISPOT) and PHPC assays. PBMC from uninfected individuals (n=5) and chronically untreated HIV-1-infected individuals in response to peptide pools representing HIV-1 Gag (n=32 samples) (A), Nef (n=24 samples) (B), and Rev (n=20 samples) (C) proteins were evaluated. Results are shown as ELISPOT and PHPC counts (net spots/million PBMC). Gag-specific ELISPOT and PHPC counts represent the summed value of net spots formed in response to each of the three HIV-1 Gag pools used for stimulation. Data from each subject were plotted individually and the horizontal line represents the respective mean number. Correlations between ELISPOT and PHPC counts (net spots/million PBMC) in response to Gag (D), Nef (E) and Rev (F) in PBMC from chronically untreated HIV-1-infected individuals are shown. Linear regression lines, correlation coefficients and p values are given in the graph.

FIG. 2. Relationship between antigen-specific IFN-γ ELISPOT (ELISPOT) or PHPC responses with plasma viral load (FIG. 2-A) or CD4 counts (FIG. 2-B) in chronically untreated HIV-1-infected individuals. Analysis was performed between plasma viremia and ELISPOT (A) or PHPC assay (B) and between CD4 counts and ELISPOT (C) or PHPC assay (D) in response to peptide pools representing HIV-1 Gag, Nef, and Rev proteins. Data are shown for log₁₀ HIV-1 RNA copies/ml plasma, CD4 cells/mm³ blood, ELISPOT and PHPC counts (net spots/million PBMC). Gag-specific ELISPOT and PHPC counts represent the summed value of net spots formed in response to each of the three HIV-1 Gag pools used for stimulation. Linear regression lines, correlation coefficients and p values are given in each graph. Significant correlation (p<0.05) is in boldface.

FIG. 3. IFN-γ ELISPOT (ELISPOT) and PHPC responses to HIV-1 Gag protein subunits in PBMC from 32 chronically untreated HIV-1-infected individuals. Schematic representation of the three HIV-1 Gag peptide pools in the complete Gag protein is shown (A). ELISPOT (B) and PHPC (C) responses to the three HIV-1 Gag peptide pools. Data are shown for ELISPOT and PHPC counts (net spots/million PBMC). Error bars represent the SD of the mean.

FIG. 4. Relationship between IFN-γ ELISPOT (ELISPOT) or PHPC responses to HIV-1 Gag subunits with plasma viral load or CD4 counts. Correlations between plasma viral load and ELISPOT (A) or PHPC assay (B) in response to Gag 17 pool, Gag 24 pool and Gag 15 pool as well as between CD4 counts and ELISPOT (C) or PHPC assay (D) in response to Gag 17 pool, Gag 24 pool and Gag 15 pool in PBMC from 32 chronically untreated HIV-1-infected individuals are shown. Correlations are shown for log₁₀ HIV-1 RNA copies/ml plasma, CD4 cells/mm³ blood, ELISPOT and PHPC counts (net spots/million PBMC). Linear regression lines, correlation coefficients and p values are given in the graph. Significant correlations (p<0.05) are in boldface.

FIG. 5. Relationship between PHPC response to HIV-1 Gag p17 subunit with plasma viral load or CD4 counts. Correlations between plasma viral load (A and C) or CD4 counts (B and D) and PHPC assay in response to Gag 17 pool in 20 PBMC samples from chronically untreated HIV-1-infected individuals in the WIHS cohort (A and B) or 52 samples (C and D) combining the WIHS cohort and the cohort described in FIG. 4 are shown. Correlations are shown for log₁₀ HIV-1 RNA copies/ml plasma, CD4 cells/mm³ blood, and PHPC counts (net spots/million PBMC). Linear regression lines, correlation coefficients and p values are given in the graph. Significant correlations (p<0.05) are in boldface.

FIG. 6. Relationship between PHPC response to HIV-1 Gag and Gag subunits (p17, p24, and p15) with changes of plasma viral load or CD4 counts over time, in four representative patients (a-d) from the WIHS cohort. Upper left panel: description of patient ID, number and timing of visits from start of follow up with corresponding viral load (log₁₀ HIV-1 RNA copies/ml plasma) and CD4 (cells/mm³) counts. Lower left panel: PHPC counts (net spots/million PBMC) against Gag p17, p24, and p15, respectively. Upper and lower right panels: linear regression lines of CD4 counts and log₁₀ VL over time, respectively.

FIG. 7. Relationship between PHPC response to HIV-1 Gag and Gag subunits (p17, p24, and p15) with changes of plasma viral load or CD4 counts over time, in 22 patients from the WIHS cohort. Linear regression lines, correlation coefficients and p values are given in the graph. Significant correlations p<0.05) are in boldface.

FIG. 8. In each cohort the upper and lower panels represent the results of the PHPC and ELISPOT responses, respectively. The surface area used for DermaVir administration in cohort 1=2 patches (0.05mg DNA per patch); cohort 2=4 patches (0.1 mg DNA per patch); cohort 3=8 patches (0.1 mg DNA per patch).

FIG. 9. After a single DermaVir Patch treatment long-lived HIV-1 Gag-specific T-cell precursors were detected in all nine HIV-1 subjects, six patients also had long-lived T-cell precursors specific to HIV-1 regulatory proteins (Rev/Tat).

FIG. 10. Cross-sectional analysis: WIHS Cohort only. This data confirms the correlation between Gag-specific PHPC responses and either plasma viremia (inverse correlation) or CD4 counts (direct correlation).

FIG. 11. Cross-sectional analysis: all 68 samples. This data shows that a range of HIV-specific PHPC responses can be correlated with a range of viral load. A statistically significant difference between individuals with high viral load/low PHPC counts versus low viral load/high PHPC counts.

FIG. 12. A longitudinal analysis shows a statistically significant difference between individuals with increased viral load/low PHPC counts versus decreased viral load/high PHPC counts over time.

### SUMMARY OF THE INVENTION

We describe here a diagnostic assay for antigen-specific T-cell responses, the steps comprising:

a. Stimulating PBMC with one or more specific antigens

b. Culturing PBMC to expand T-cells

c. Restimulating expanded T-cells with one or more specific antigens

d. Measuring cytokine release.

This test is demonstrated for the first time on a virus, namely HIV-1, where there is a long-recognized need to characterize a patient's immunological status. In this assay, the antigens may be multiple peptide pools, as demonstrated in our examples of the Gag protein, or a single peptide pool as in the examples for the Rev and Tat genes, but the antigen could also be a single peptide. In the present application, peptide pools from the Gag, Nef and Rev proteins have been studied. For HIV-1, Gag protein subgroup p17 and/or p24 gave particularly good correlations with viremia and CD4 counts, two well-recognized markers for disease progression.

This assay relies on extended stimulation of PBMC for at least 2 days, preferably 7-21 days, more preferably about 7-14 days.

To develop the assay we have started with a chronic infection that represents a world emergency: HIV infection. An advantage of this assay is that it removes a major barrier to vaccine development for chronic infections. In this disease, vaccine development has been hampered for the past 25 years due to the absence of a test that can correlate immune system status with disease progression. Here we describe a test that yields a quantitative determination of an HIV-specific T cell population that can predict disease progression in HIV infection.

Another advantage of the invention is that it modifies the standard IFN-γ ELISPOT (ELISPOT) assay, which did not yield any correlation between immune responses (ELISPOT counts) and HIV-1 viral load, so that it quantifies another subpopulation of T-cells that we have suspected to play an important role in control of infectious diseases: the precursor T-cells that can proliferate and differentiate to HIV-specific cytotoxic T-cells. The advantages of this invention are obtained by culturing PBMC specimens isolated from HIV-1 infected patients in the presence of HIV antigens for several days ( preferably 2-21 days, more preferably 7-14 days) to allow precursor T-cells to expand in response to antigen exposure and to acquire effector function. During the preferred culture time, the antigen-stimulated IFN-γ producing effector T-cells (the population determined with test using short-term antigen stimulation, e.g. ELISPOT) would be expected to undergo apoptosis, or normal cell death. After the several days of culture, the functionality of the expanded T-cells is tested after restimulation with HIV antigens with the standard ELISPOT assay to obtain a quantitative measurement of cytokine release (spot counts). Therefore, our new assay quantifies a T-cell population that we called antigen-specific precursor T-cells with high proliferative capacity (PHPC count). PHPC counts provide a measurement of T-cell precursors that can differentiate to antiviral cytotoxic T-cells and eliminate antigen-presenting infected cells. These cells might be similar to the long lasting memory T-cells characterized by several phenotypic markers. Our results definitely determined that the PHPC assay detects a T-cell population that is quantitatively and qualitatively different from the T-cells detected by the ELISPOT assay.

To investigate the differences between the newly developed PHPC assay and the standard ELISPOT we tested the PBMC from 32 chronically untreated HIV-1-infected individuals with both assays. Surprisingly, we found that a high magnitude of HIV-specific PHPC counts, but not ELISPOT counts, significantly correlated with low plasma viremia. Analysis of 20 additional PBMC samples from an independent cohort of chronically untreated HIV-1-infected individuals confirmed the correlation between HIV-specific PHPC response and either plasma viremia (inverse correlation) or CD4 counts (direct correlation). These results demonstrate that the quantity of antigen-specific T-cells detected with our PHPC test can be used as diagnostic tool to determine the immunological status of chronically-infected patients. In addition, a certain range of antigen-specific PHPC counts can be correlated with a certain range of viral load (“VL”). For example, Gag-specific PHPC above 3-6,000 counts were associated with VL lower than 3-4 logs, and p17-specific PHPC above 1-3,000 counts were associated with VL lower than 3-4 logs.

In a longitudinal analysis 22 individuals participating in the Women's Interagency HIV Study, US, were monitored biannually for >10 years. In this longitudinal cohort HIV-specific PHPC counts inversely correlated with subsequent VL change/year. Also in this case a certain range of antigen-specific PHPC counts can be correlated with a certain range of viral load change. For example, Gag-specific PHPC above 4-7,000 counts and/or p17-specific PHPC above 1-3,000 counts were associated with no increase or decrease of VL during >10 follow up. The result demonstrates that PHPC counts not only detect the present immunological status of a chronically-infected patient but also offer a forecast on the evolution of the disease. High PHPC counts correlates with low viral load and high CD4 counts and predict stable viral load over time. These data an additional advantage of the PHPC assay, that is to be used as a diagnostic tool to predict the disease progression via the immunological status of chronically infected patients. To our knowledge there is no test in the prior art that can predict the disease progression of chronically-infected patients in any chronic diseases.

The PHPC test can be used as a diagnostic tool as well as a tool to monitor changes in the antiviral immune responses induced by immune therapies or vaccines. The development of immune-based therapies and vaccines has been hampered to date due to the absence of known immune correlates between antigen-specific T-cell responses and disease progression. For example, the DermaVir Patch Immune Therapy has been developed to treat HIV-infected individuals by induction of long-lasting antiviral HIV-specific T-cells able to kill HIV-infected cells and consequently suppress viral replication (PCT US97/02933 ‘Methods and Compositions for Therapeutic and Genetic Immunization’, U.S. Pat. No. 6,420,176 ‘Method of Delivering Genes into Antigen Presenting Cells of the skin’, U.S. Pat. No. 7,196,186 “Plasmid DNA and Uses therefore”). The DermaVir Patch treatment decreased viral load in chronically-infected monkeys, and similar data needs to be still shown in chronically-infected human subjects. The conventionally used ELISPOT counts did not correlate with either decreased viral load or the PHPC counts in HIV-infected individuals. However, we have shown here a dose dependent increase of the magnitude and breadth of HIV-specific PHPC counts in all DermaVir Patch treated HIV+ patients to levels equal or superior to those measured in chronically HIV-infected, untreated individuals whose immune system was able to control viral load. Based on the available data described in this patent application we predict that DermaVir Patch will improve disease progression compared to placebo patch treated patients, as it did in monkeys.

The PHPC assay provides an immune diagnostic tool for patients suffering in chronic diseases to (1) determine their specific immunological status; (2) to predict their individual disease progression; (3) for individual patient management (for example for determining when to start therapy or how intensely to monitor a patient); and (4) for the design, testing and screening of therapies based on immune modulation, targeted immune amplification, and prophylactic and/or therapeutic vaccines.

### DETAILED DESCRIPTION OF THE INVENTION

Although several lines of evidence suggest that antigen-specific CD8⁺ T-cell responses play a critical role in controlling HIV infection in humans and SIV in the macaque model, the precise immune correlates of HIV control remain to be identified.

The IFN-γ ELISPOT assay (ELISPOT) is currently used to identify HIV-specific T-cell responses in humans. This assay quantifies T-cells secreting IFN-γ within 18-24 hours of antigen stimulation, that is, short-lived effector T-cells. Conversely, the present invention quantifies precursors to antigen-specific T-cells having high proliferative capacity (PHPC assay), likely representing memory T-cells. In this assay, cells are first cultured with the desired antigen for at least 2 days, preferably 7-21 days, in order to allow precursor T-cells to expand in response to antigen and to acquire effector function. In the meantime, the antigen-stimulated effector T-cells should undergo apoptosis.

In the present application, antigen-specific T-cells quantified by ELISPOT and PHPC assays were compared in order to discern which responses correlate with viral control and preservation of CD4⁺ T-cells in chronically HIV-infected individuals naïve to antiretroviral treatment. We focused on immune responses to Gag and Nef proteins because they are the most frequently recognized by HIV-infected individuals and to Rev protein because it is expressed early in the virus life cycle and Rev-specific immune response is present in long-term asymptomatic HIV-infected patients. Subsequently, we analyzed the relationship between antigen-specific IFN-γ production, measured by both ELISPOT and PHPC assays, and plasma viral load or CD4 cell counts and found that antigen-specific PHPC (particularly against Gag p17), but not ELISPOT, response is associated with low plasma viremia and high CD4 cell counts.

The present assay is the first to demonstrate that Gag-specific responses inversely correlated with the magnitude of plasma viral load and directly correlated with CD4⁺ T-cell counts in chronically HIV-infected individuals naïve to antiretroviral treatment. The results indicate that the presence of antigen-specific T-cell precursors with high proliferative capacity is associated with HIV control and preservation of CD4 counts, hallmarks of slow progression to AIDS, similar to what has been recently observed in a non-human primate model.

HIV-1 Gag p17 contains many overlapping CTL epitopes restricted by several HLA molecules. The significant association between Gag PHPC response directed toward the p17 subunit and low viral load is consistent with the observations that HLA-A2-restricted CD8⁺ T-cell responses against an epitope in p17 (aa 77-85, SLYNTVATL), which is presented in high abundance in chronically HIV-1-infected cells, inversely correlate with viral load, as measured by tetramer binding staining, and has been associated with long-term control of HIV. Results from a recent study provides evidence that Gag-derived epitopes are the first to be presented in infected lymphocytes and that the early presentation of Gag-derived epitopes does not require de novo protein synthesis. Because HIV-1 products are translated sequentially as p17, p24 and p15, it can be hypothesized that p17 is presented earlier than the other Gag subunits. Additionally, here we report a significant association between Gag p17-specific PHPC counts and high CD4⁺ T-cell counts. Altogether, our data suggest an important role of Gag p17-specific PHPC responses in control of viremia.

The finding that the mean antigen-specific IFN-γ responses detected by the PHPC assay was higher than those detected by the ELISPOT assay suggests a higher sensitivity of the PHPC assay, however, we did not find any significant correlation between both IFN-γ ELISPOT assays in response to Gag, Nef, and Rev, in agreement with other investigators. These results indicate that different IFN-γ producing cell populations are being measured, reflecting the different nature of these two assays. In fact, during the 12-day culture period, T-cell precursors expand in response to the antigen and differentiate into effectors cells, while the immediate effector cells present in the culture undergo apoptosis.

The ELISPOT assay is widely used to identify antigen-specific T-cell responses in both natural HIV infection and following immunization with vaccine candidates. Concordant with studies published by others, we found that the mean Gag and Nef-specific IFN-γ responses were higher than the mean Rev-specific response by using the ELISPOT assay. However, several evidences indicate that antigen-specific responses measured by the ELISPOT assay have no direct effect on plasma viral load or CD4 counts. Only the broadness of the ELISPOT response inversely correlated with plasma viremia in the setting of HIV infection, however, this demonstration required the observation of large cohorts. It is therefore noteworthy that a small number of patients was sufficient to demonstrate a robust inverse correlation between the immune responses measured by PHPC assay and plasma viral load.

An inverse correlation between HIV-specific CTL responses and plasma viremia had been obtained in the past by the measurement of antigen-specific CTL activity of in vitro expanded PBMC by the chromium-release assay. The PHPC assay follows basic principles similar to those of the classical chromium-release CTL assay. The PHPC method not only magnifies the immune response by allowing the antigen-specific T-cells present to divide, but also enables the resting memory T-cells to differentiate into effector cells. We demonstrated in our study that cells with high proliferative capacity are associated with suppression of viral replication. As such, the PHPC assay provides an alternative to the laborious in vitro re-stimulation CTL assay, allowing the evaluation of antigen-specific precursor T-cell responses in an ELISPOT format.

In a longitudinal cohort study the magnitude of Gag, p17- and p24-specific PHPC counts inversely correlated with subsequent VL change/year, indicating that PHPC counts can predict stable control of viral load over time, up to almost 10 years follow up. A trend, although not significant, was found for a direct correlation between PHPC counts and changes of CD4 counts over the same time period.

By using an immune therapy approach, namely DermaVir, PHPC responses were induced by a single DermaVir Patch treatment of 9 out of 9 HIV-infected individuals. DermaVir Patch immunization using 0.4 and 0.8 mg DNA induced 10-100 fold more precursor T-cell counts than using 0.1 mg DNA. ELI SPOT responses were induced only in 5/9 patients. T-cell responses measured by ELISPOT do not seem to have any relevance in either viral load suppression or protection against infection. One year after the single immunization HIV-specific precursor T-cells were still present (10-1000 fold compared to baseline), albeit in lower frequency than 28 days after treatment, suggesting that the precursor T-cells induced by this immunization have long half-life. Single immunization broadened the specificity of HIV-specific precursor T-cells. T-cell responses were induced against both structural and regulatory proteins suggesting that the novel DNA plasmid in the product is a potent immunogen.

In conclusion, our finding represents the first evaluation of HIV-specific memory T-cells with high proliferative capacity quantified by the PHPC assay in HIV-infected individuals naïve to antiretroviral treatment. The presence of T-cells with high proliferative capacity is associated with low plasma viremia and high CD4⁺ T-cell counts.

The PHPC assay will be useful to evaluate the status of the immune system and the recovery of its function in patients treated with antiretroviral drugs as well as to assess the immunogenicity of prophylactic and therapeutic vaccines.

### EXAMPLES

The following examples illustrate the practice of various aspects of the present inventions. They do not limit the inventions, or the claims, which follow them.

### Example 1

### Individual Characteristics and Samples Analyzed

A total of 32 chronically HIV-1-infected individuals naïve to antiretroviral treatment and 5 uninfected control subjects were analyzed. All samples from HIV-1-infected individuals were obtained from stored frozen PBMC: 28 samples were from the HIV/AIDS Outpatient Clinic, Fondazione Istituto di Ricovero e Cura a Carattere Scientifico Policlinico San Matteo, Pavia, Italy, and 4 samples were from the McGill University Health Center, Montréal, Canada. Table I provides the viral load and CD4⁺ T-cell counts for the 32 HIV-1-infected individuals.

TABLE I
Viral load and CD4 counts in chronically HIV-1-infected
individuals naïve to antiretroviral treatment

| Individual | Plasma viral load^{a} | CD4 counts^{a} |
|---|---|---|
| ID | (HIV RNA copies/ml plasma) | (cells/mm³) |
| 1 | 12,609 | 629 |
| 2 | 15,082 | 311 |
| 3 | 17,124 | 389 |
| 4 | 24,285 | 257 |
| 5 | 38,053 | 623 |
| 6 | 40,999 | 716 |
| 7 | 67,700 | 283 |
| 8 | 77,283 | 337 |
| 9 | 79,597 | 136 |
| 10 | 122,905 | 367 |
| 11 | 179,769 | 87 |
| 12 | 210,987 | 141 |
| 13 | 303,617 | 36 |
| 14 | 312,628 | 200 |
| 15 | 89,640 | 177 |
| 16 | 129,000 | 150 |
| 17 | 40,000 | 68 |
| 18 | 409,764 | 41 |
| 19 | 87,171 | 411 |
| 20 | 499 | 189 |
| 21 | 19,631 | 210 |
| 22 | 145,555 | 210 |
| 23 | 529 | 600 |
| 24 | 749 | 1,218 |
| 25 | 86,120 | 38 |
| 26 | 14,000 | 589 |
| 27 | 13,834 | 323 |
| 28 | 6,263 | 345 |
| 29 | 55,282 | 441 |
| 30 | 8,559 | 295 |
| 31 | 8,698 | 1,122 |
| 32 | 59,196 | 290 |
| Mean | 83,660 | 351 |
| SD | 101,136 | 283 |

^{a}At the time of ELISPOT analyses

The 5 uninfected subjects were recruited from the blood bank at the Fondazione Istituto di Ricovero e Cura a Carattere Scientifico Policlinico S. Matteo, Pavia, Italy.

Twenty additional samples from chronically untreated HIV-1-infected individuals were obtained from the Women's Interagency HIV Study (WIHS) cohort, Georgetown University Medical Center participating site. At the time of PHPC analysis, the mean plasma viral load was 11,528±21,527 HIV RNA copies/ml (range: 80-71,000) and the mean CD4 counts was 595±370 cells/mm³ blood (range: 230-1,537). Peripheral blood mononuclear cells (PBMC) were isolated by standard Ficoll-Hypaque centrifugation and cryopreserved in fetal bovine serum (FBS-Gibco) containing 10% dimethyl sulphoxide (DMSO-Sigma-Aldrich) and kept in liquid nitrogen until use. PBMC were thawed, washed and rested overnight at 37° C., in 5% CO₂ atmosphere, in RPMI 1640 medium (Eurobio) containing 2 mM L-glutamine (Eurobio) and supplemented with 10% heat-inactivated FBS (Gibco), 100 IU/ml penicillin and 100 μg/ml streptomycin (Eurobio) (complete culture medium). One day later, cell viability determined by trypan blue exclusion was ≧85%.

### Synthetic Peptides

Peptides used in this study were obtained from the National Institutes of Health (NIH) AIDS Research and Reference Reagent Program, Division of AIDS, NIAID, NIH. Peptides (15 aa in length with 11-aa overlap) corresponded to the complete sequence of HIV Consensus B Gag, Nef, Tat, and Rev. Gag peptides (n=123) were divided into three pools (41 peptides per pool): Gag p17 pool spanned all p17 and the first 43 amino acids of p24, Gag p24 pool spanned mainly p24, while Gag p15 pool spanned the last 35 amino acids of p24 and all p15. Nef (n=49), Tat (n=23), and Rev (n=27) peptides were used as a single pool. Peptides were prepared as the corresponding pool at a concentration of circa 100 mg/ml in DMSO, aliquoted and stored at −20° C.

### IFN-γ ELISPOT Assay (ELISPOT)

A human IFN-γ ELISPOT kit (Diaclone) was used. MultiScreen™-IP 96-well plates (Millipore) were coated with capture monoclonal antibody (mAb) diluted 1/100 in PBS and incubated overnight at 4° C. After several washes with PBS, plates were blocked for 2 h at room temperature with complete culture medium. PBMC were added in duplicate at an input cell number of 1×10⁵ cells per well in 100 μl complete culture medium. HIV peptide pools were diluted 1/200 in complete culture medium and 100 μl was added to each well. Phytoemagglutinin (PHA) (5 μg/ml; Sigma-Aldrich) was used as a positive control. Cells resuspended only in complete culture medium served as a negative control. After an incubation of 24 h at 37° C. 5% CO₂ atmosphere, plates were washed with phosphate buffered saline (PBS) supplemented with 0.1% Tween20 (Sigma) (wash buffer) followed by an overnight incubation at 4° C. with 100 μl per well biotinylated detection mAb, diluted 1/100 in PBS supplemented with 1% bovine serum albumin (BSA-Diaclone) (PBS +1% BSA). Plates were washed with wash buffer and 100 μl per well strepatavidin-alkaline phosphatase conjugated, diluted 1/1000 in PBS +1% BSA, was added and incubated at 37° C. 5% CO₂ atmosphere for 1 h. The wells were then washed with wash buffer, and 100 μl substrate buffer (BCIP/NBT; Diaclone) was added per well. The colorimetric reaction was terminated after 10 min at room temperature by washing several times with tap water. Plates were air-dried and the spots counted using an automated ELISPOT reader system (A-EL-Vis). The mean background medium control response was 2.3 (±3.4) IFN-γ spots per well. The mean number of IFN-γ spots per well in response to PHA stimulation was 334.2 (±186).

T-Cell Precursors with High Proliferative Capacity (PHPC) Assay

PBMC (5×10⁵ per ml) were plated in each well in a 24-well flat-bottom tissue culture plate. Cells were stimulated with HIV peptide pools (diluted 1/400, one pool per well), or PHA (5 μg/ml) or complete culture medium (control wells) and cultured at 37° C. 5% CO₂ atmosphere for 12 days. On days 3 and 7,500 μl of supernatant per well were removed and replaced with fresh complete culture medium supplemented with 10 IU/ml recombinant human interleukin-2 (IL-2-R&D Systems). On day 11 cells from each well were counted, seeded on an ELISPOT plate to be restimulated with the same HIV peptide pools, and, on day 12, cells were washed three times with complete culture medium and tested, at 100 μl per well (2.5×10⁴ cells, in duplicate or triplicate) in the same way as the ELISPOT, measuring cytokine release in response to the corresponding antigen used for stimulation. (Spot formation). Spots were counted using the same automated ELISPOT reader system and set-up parameters as for the ELISPOT assay. The mean number of IFN-γ spots per well in response to control medium was 10.3 (±14.1). A mean number of 103.3 (±92.5) IFN-γ spots per well in response to PHA stimulation was detected.

### Data Analysis

The mean number of spots from duplicate or triplicate wells was adjusted to 1×10⁶ PBMC. Data are presented as ELISPOT counts and PHPC counts. The PHPC counts (net spots/million PBMC) were calculated as follows: (mean number of spots/million PBMC in wells from each pool of peptides minus mean number of spots/million PBMC in wells with control medium)×proliferation index. The proliferation index was calculated as number of antigen-stimulated cells after 12 days of culture divided the number of control (medium-stimulated) cells after 12 days of culture, in order to account for the fact that the PHPC assay allows for the expansion of T-cells during the culture. ELISPOT and PHPC counts in response to Gag p17 pool, Gag p24 pool and Gag p15 pool were summed to calculate total Gag response (denoted as Gag). Statistical analysis and scatterplot graphic representations were performed using Statistica software (Statistica for Windows version 7.1). Student's t-test was used to assess differences in net spots/million PBMC detected by ELISPOT and PHPC assays. Correlations between antigen-specific responses determined by ELISPOT and PHPC assays with plasma viral load and CD4 counts were determined by Pearson's test. A p value<0.05 was considered statistically significant.

### Results

### Detection of HIV-Specific IFN-γ Production by ELISPOT and PHPC Assays

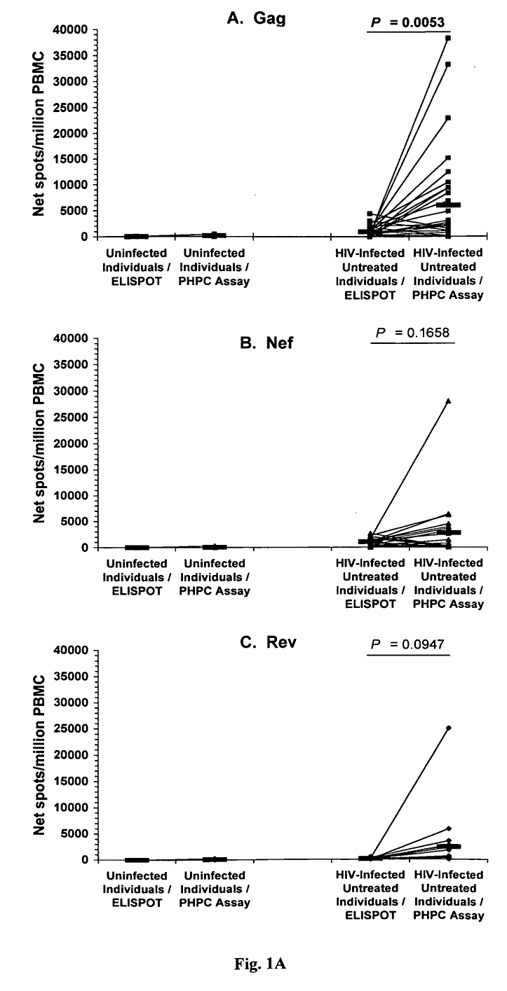

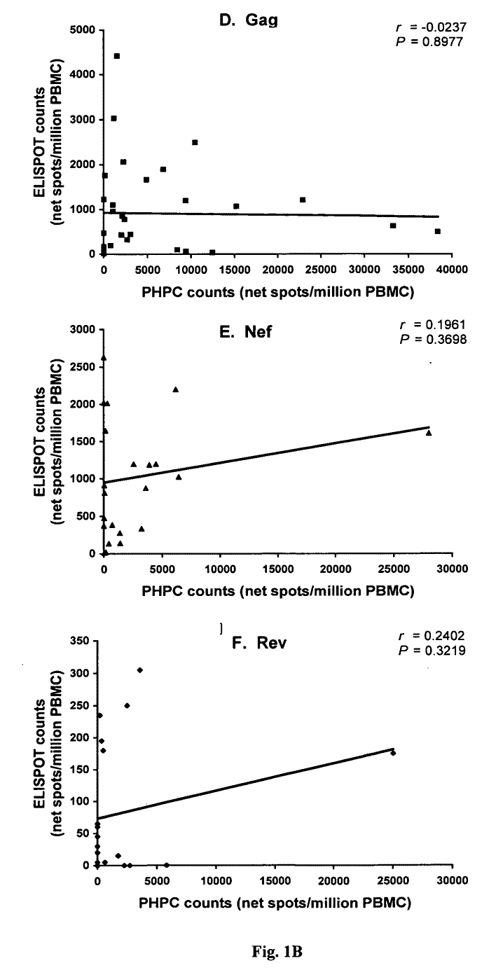

Using PBMC specimens from 32 chronically HIV-infected individuals naïve to antiretroviral treatment and five uninfected control subjects, ELISPOT and PHPC counts were evaluated in response to peptide pools representing the complete HIV Consensus B Gag, Nef and Rev proteins. Based on the available number of PBMC from HIV-infected individuals, T-cell responses to Gag was evaluated in all 32 samples, while Nef and Rev responses were evaluated in 24 and 20 samples, respectively; 18 samples were tested for all three proteins. In HIV-infected individuals the mean number of ELISPOT counts (net spots/million PBMC) in response to Gag, Nef, and Rev was 913.4 (SD: ±1021.7), 1031 (±770.7), and 105.8 (±141.5), respectively (FIG. 1A-C). The mean number of PHPC counts (net spots/million PBMC) in response to Gag (6005.2±9519.1 spots/million PBMC), Nef (2747.3±5878.6 spots/million PBMC), and Rev (2386.7±5715.2 spots/million PBMC) was 7-fold (p=0.0053), 3-fold (p=0.1658), and 23-fold (p=0.0947) higher than those detected by the ELISPOT assay, respectively (FIG. 1A-C).

The PHPC results did not simply reflect a proportional increase of the ELISPOT counts. In fact, in some patients the number of spots obtained by PHPC assay was lower than that obtained by ELISPOT assay. To confirm that the PHPC assay was not just an “ELISPOT assay with increased sensitivity” we examined the relationship between the two assays. No correlation was found between ELISPOT and PHPC counts in response to Gag, Nef, and Rev (p=0.8977, p=0.3698, and p=0.3219, respectively) (FIG. 1D-F). Restricting the analysis to those 18 samples that have been tested for all three proteins gave similar results (data not shown). These results suggest that the PHPC assay is not only quantitatively but also qualitatively different from the ELISPOT assay, confirming that different T-cell populations are being evaluated.

Gag-Specific PHPC Response Correlates with Low Viremia and High CD4 Counts

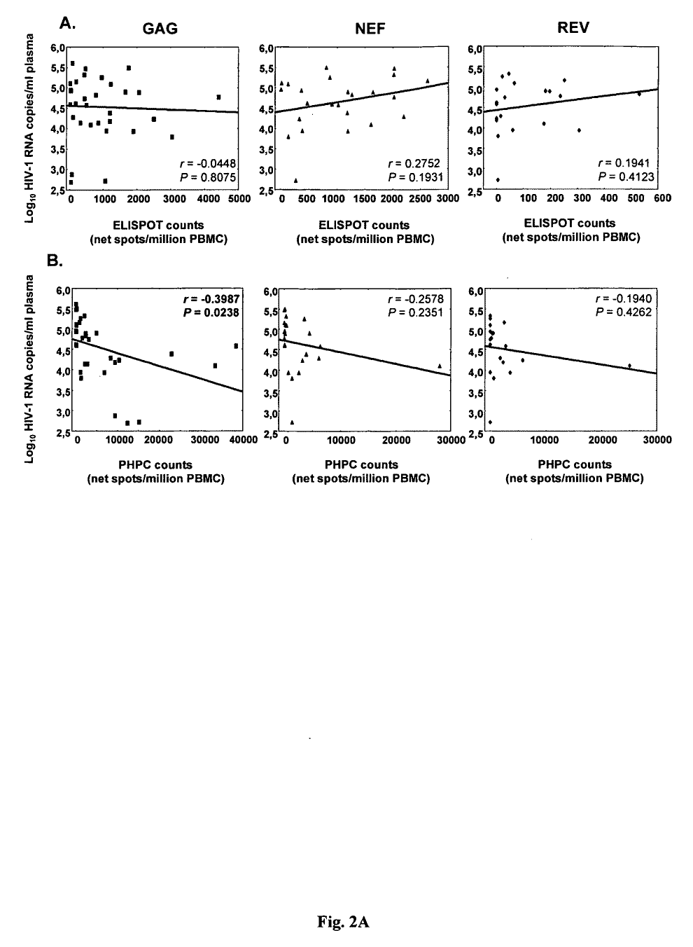

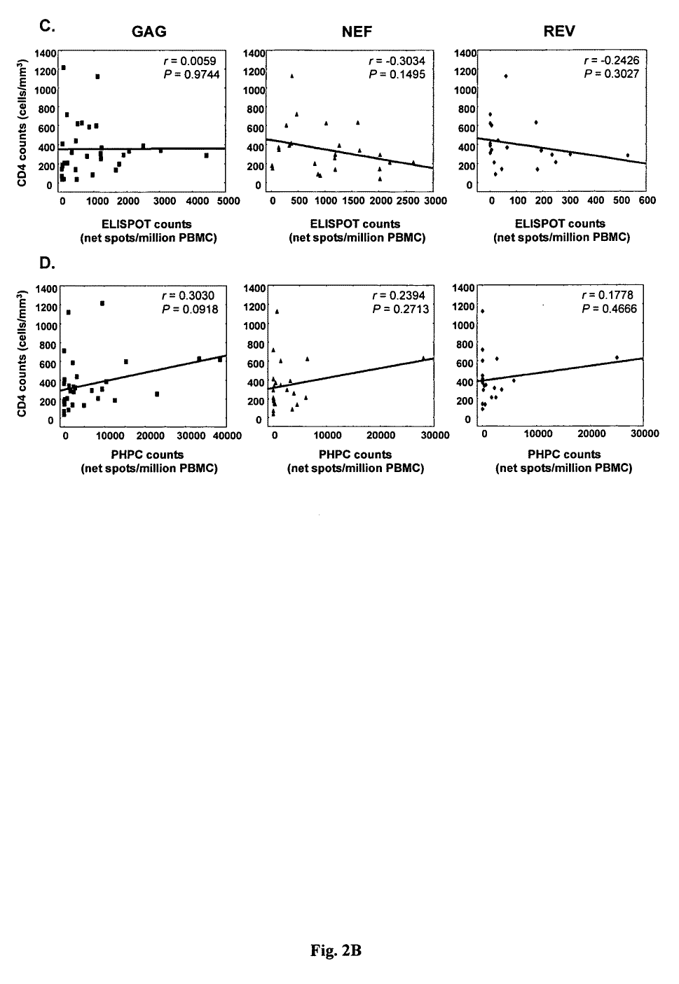

We then investigated the association between antigen-specific ELISPOT and PHPC responses and the level of plasma viremia in chronically untreated HIV-infected individuals. As shown in FIG. 2A, no statistically significant association was found between Gag-, Nef-, and Rev-specific ELISPOT counts and plasma viral load. Conversely, a significant negative correlation between Gag-specific PHPC counts and plasma viral load was found (p=0.0238) (FIG. 2B).

Next, the association between antigen-specific ELISPOT and PHPC responses and CD4 counts in chronically untreated HIV-infected individuals was analyzed. No statistically significant association was found between Gag-, Nef-, and Rev-specific ELISPOT counts and CD4 counts (FIG. 2C); however, a positive trend was observed between Gag-specific PHPC counts and CD4 counts (FIG. 2D). Altogether, the results demonstrate that Gag-specific T-cell PHPC are associated with low viral replication and increase of CD4 counts.

The importance of antigen-specific helper CD4 T-cells in vivo for sustained memory CD8 T-cell responses during chronic infections has been pointed out previously. To determine whether CD4 help is also required for optimal responses in the PHPC assay and to establish whether the correlation between the PHPC assay and CD4 counts simply reflected reduced CD4 help in the assay, we compared Gag-specific PHPC response in total and CD4-depleted PBMC from three chronically untreated HIV-infected individuals. We found comparable responses upon CD4 cell depletion, suggesting that Gag-specific T-cell response detected by PHPC assay is likely independent from CD4 help (data not shown).

### ELISPOT and PHPC Responses to HIV Gag Protein Subunits

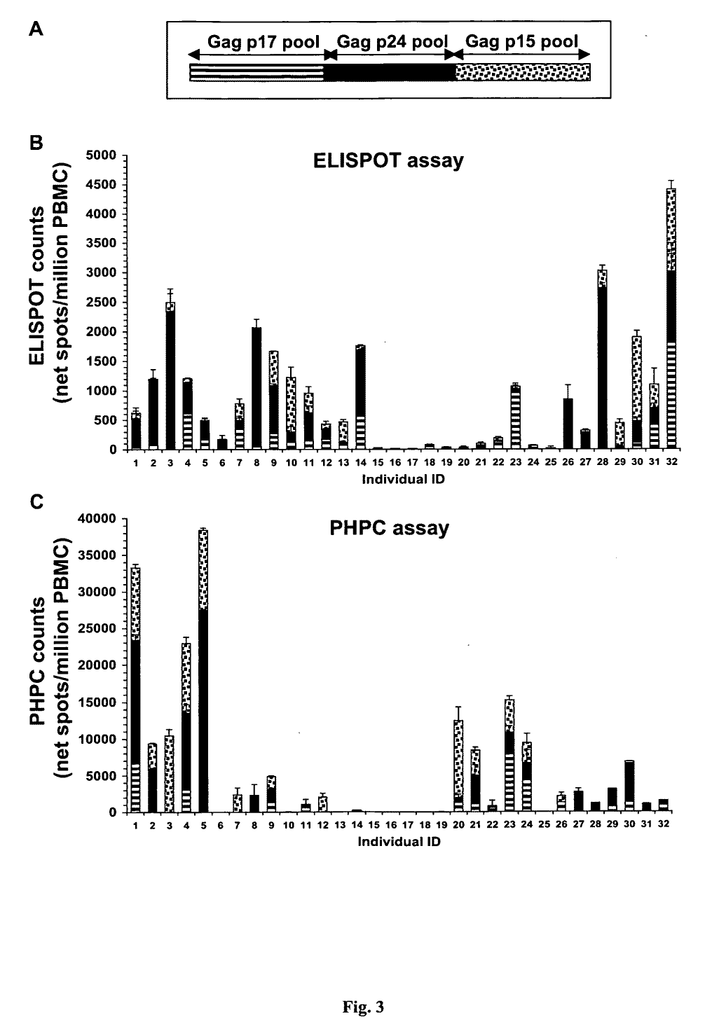

To further characterize the HIV-specific Gag response, we dissected ELISPOT and PHPC responses to the three HIV Gag peptide pools, spanning mainly p17, p24 and p15 (FIG. 3A). Gag-specific ELISPOT and PHPC responses (FIGS. 3B and C, respectively) did not mirror each other in either magnitude or distribution; some subjects who responded in the PHPC assay did not respond in the ELISPOT assay and vice versa, further illustrating that these assays are measuring different T-cell responses with distinct specificities.

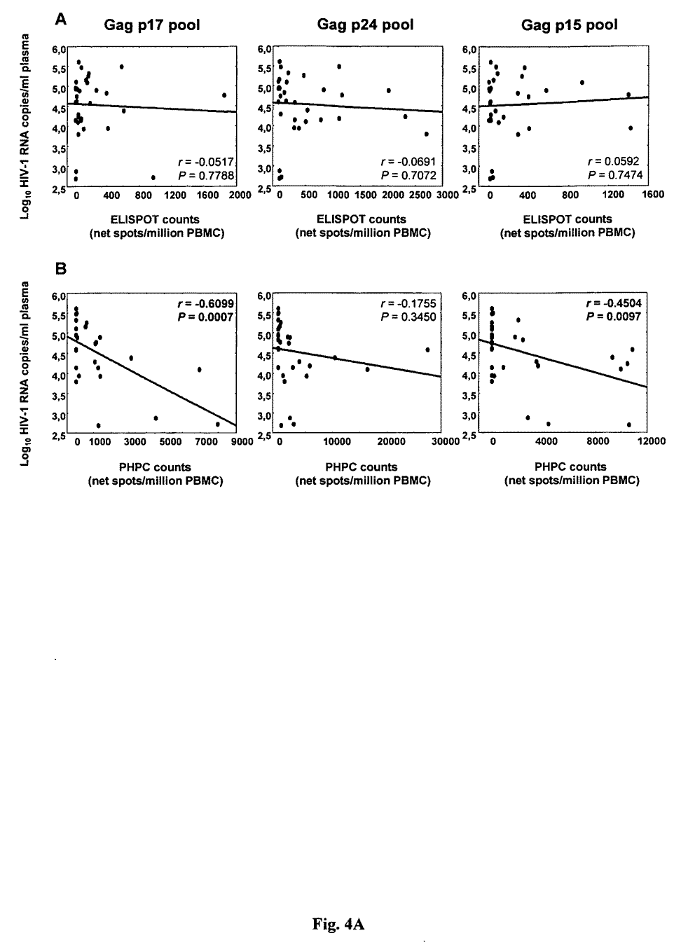

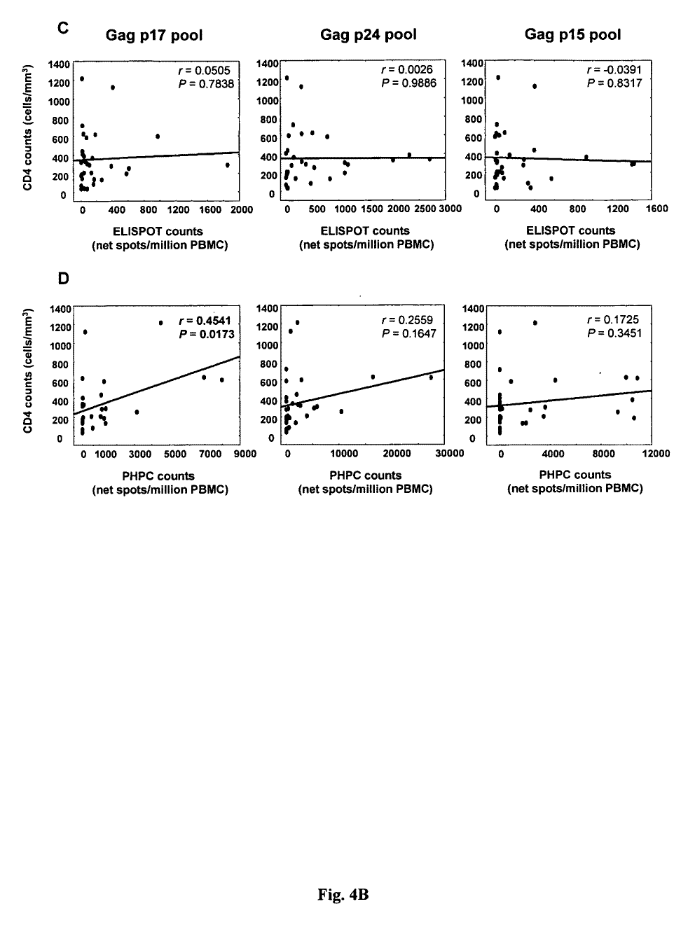

No statistically significant correlation was found between ELISPOT counts in response to the three HIV Gag peptide pools and plasma viremia (FIG. 4A); in contrast, p17-specific and p15-specific PHPC counts significantly correlated with low plasma viral load (p=0.0007 and p=0.0097, respectively) (FIG. 4B).

No statistically significant association was observed between ELISPOT responses to the three HIV Gag peptide pools and CD4 counts (FIG. 4C). However, when PHPC responses to the three Gag peptide pools were plotted against CD4 counts, only p17-specific PHPC and CD4 counts significantly and directly correlated (p=0.0173) (FIG. 4D).

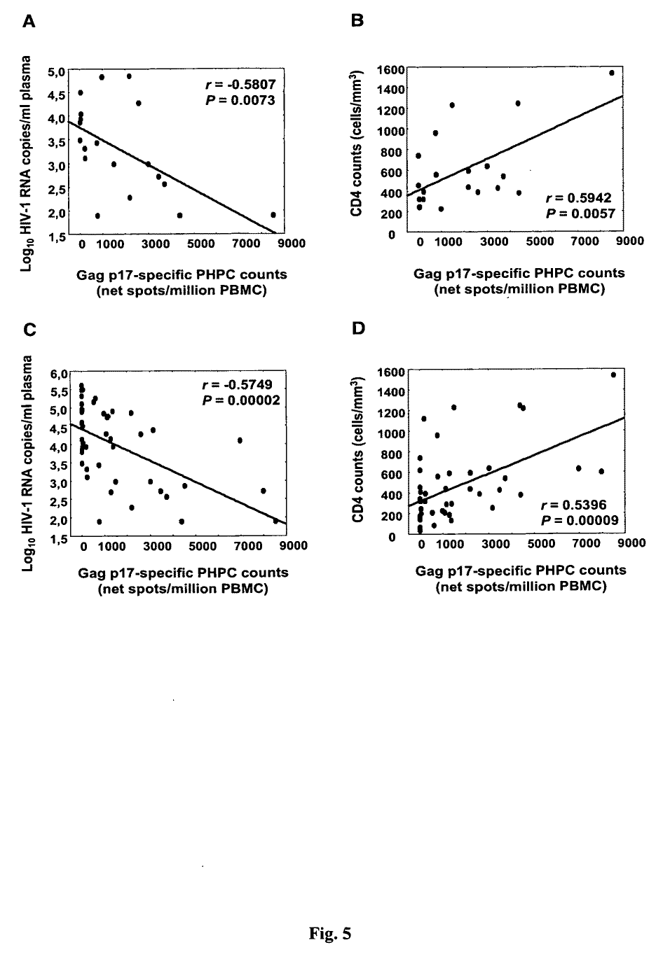

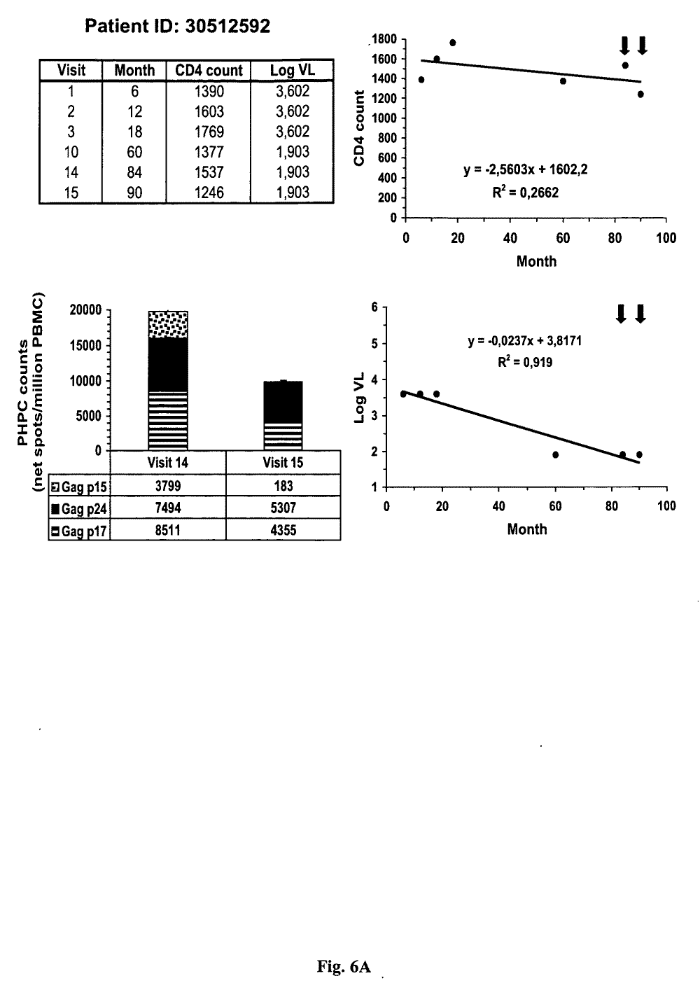

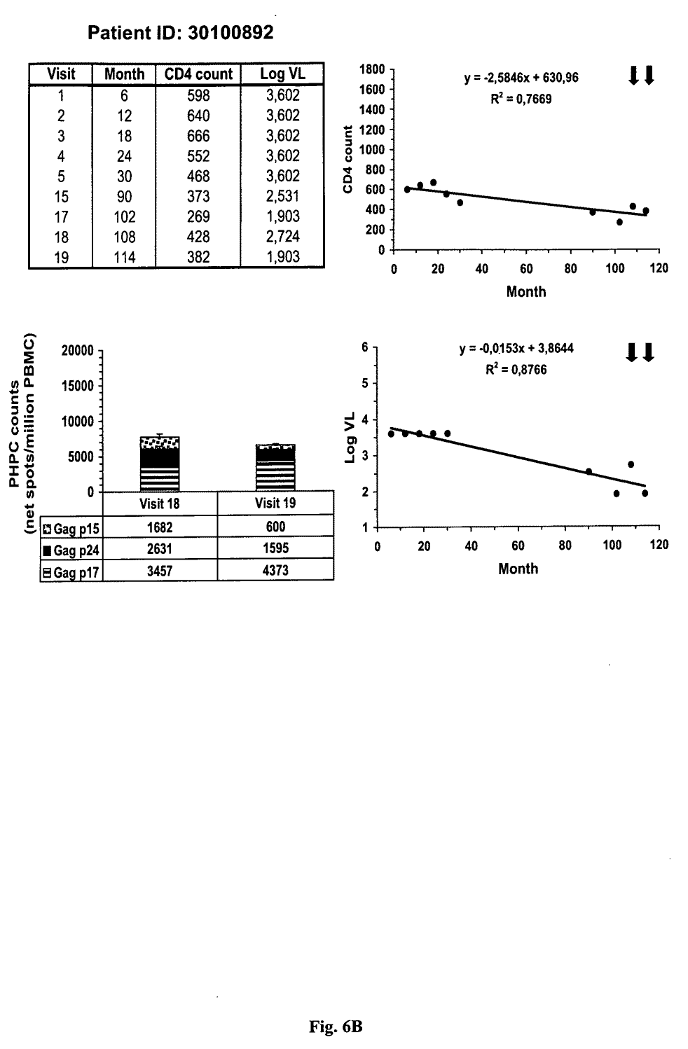

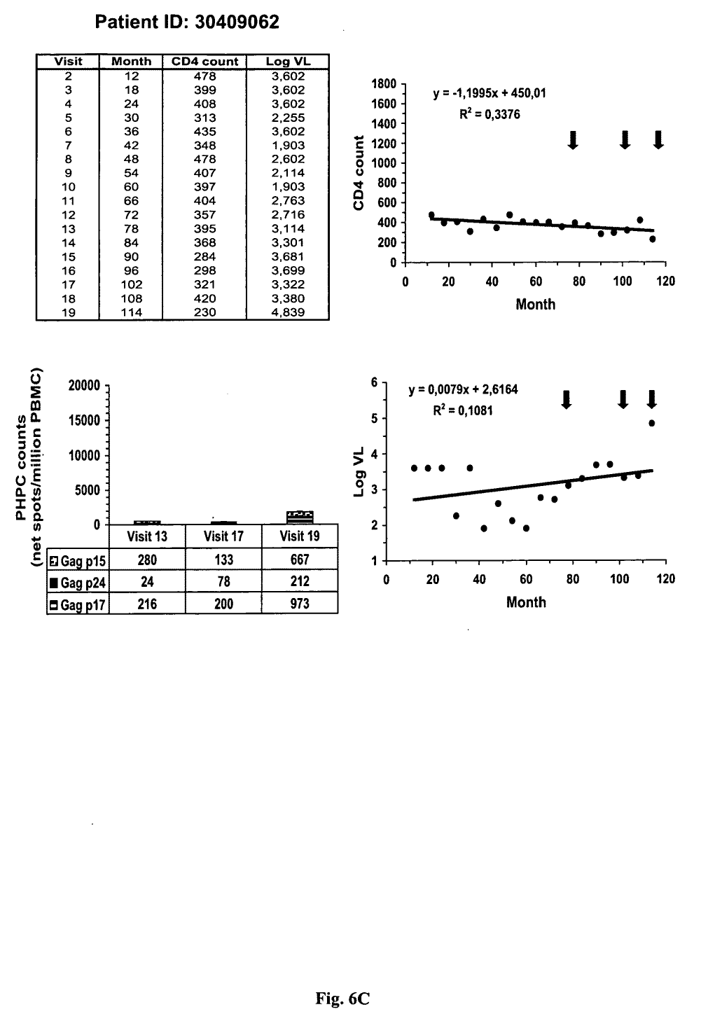

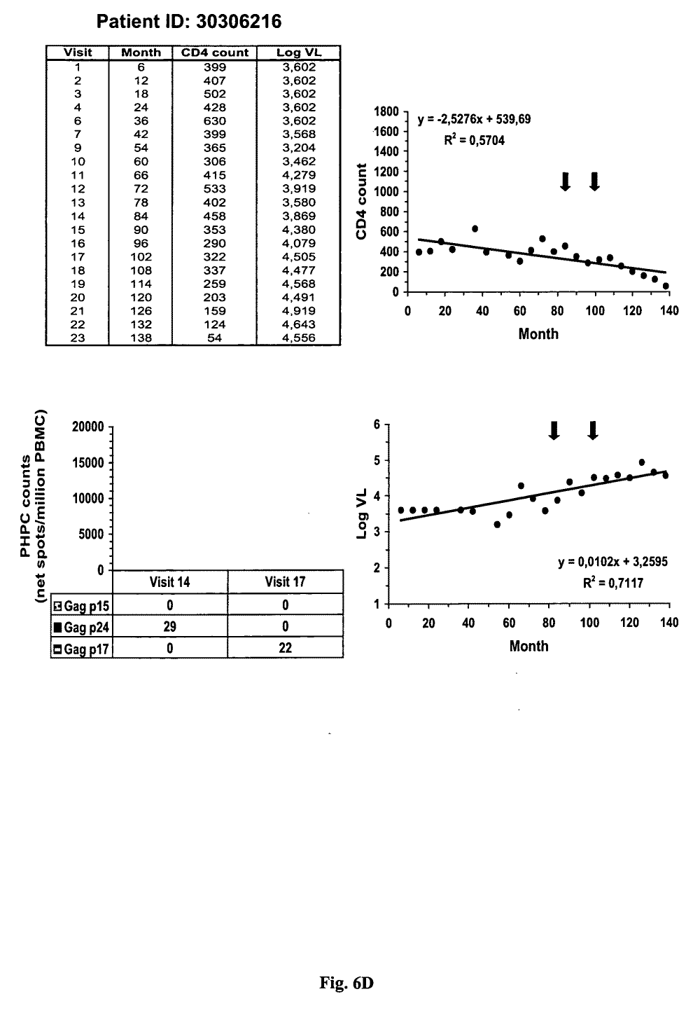

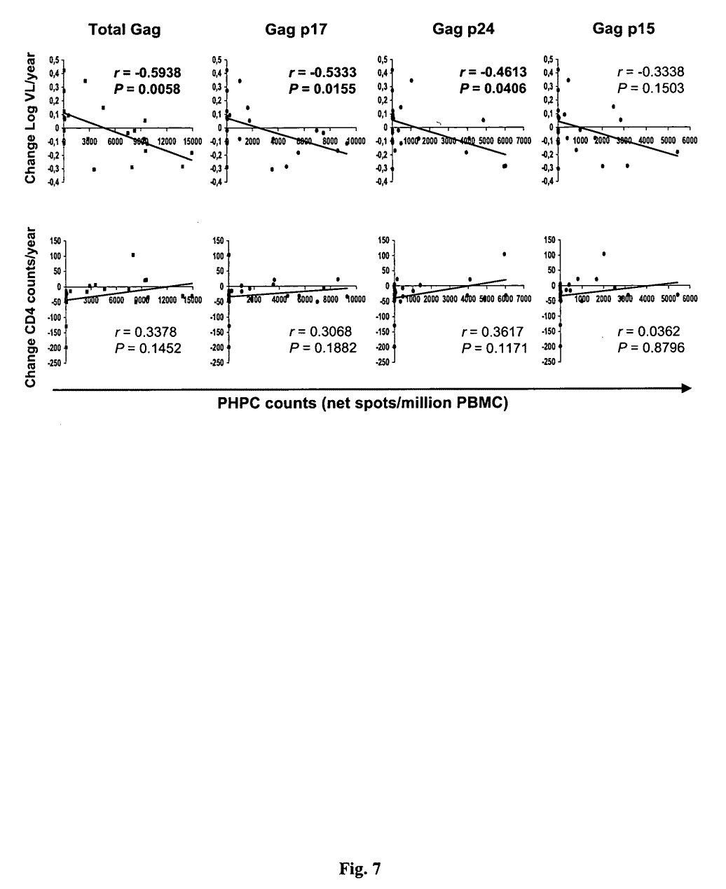

Since the association between p17-specific PHPC response and plasma viral load was the most significant one, we evaluated p17-specific PHPC response in 20 additional samples from a separate cohort of chronically untreated HIV-infected individuals participating in the Women's Interagency HIV Study (WIHS) cohort (Georgetown University participating site). The inverse correlation between p17-specific PHPC response and plasma viremia was confirmed (r=−0.5807, p=0.0073, FIG. 5A). The correlation between p17-specific PHPC response and CD4 counts was also confirmed (r=0.5942,p=0.0057, FIG. 5B).

Combining the two cohorts (total samples=52) further increased the statistical significance of the inverse correlation between plasma viremia and p17-specific PHPC responses (p=0.00002, FIG. 5C) and the direct correlation between CD4 counts and p17-specific PHPC responses (p=0.00009, FIG. 5D).

### Example 2

### Individual Characteristics and Samples Analyzed

A total of 22 comparable individuals participating in the Women's Interagency HIV Study (WIHS), US, were monitored biannually for a median of 11.5 (range 1.5-12.5.5, average 9.7, SD 3.6) years.

### Synthetic Peptides, PHPC Assay, and Data Analysis

If not indicated, synthetic peptides, PHPC assay, and data analysis were the same as in Example 1. Changes/year of CD4 count and plasma viremia (VL) were calculated using a linear regression model.

### Results

In a longitudinal analysis 22 comparable individuals participating in the Women's Interagency HIV Study, US, were monitored biannually for a median of 11.5 (range 1.5-12.5.5, average 9.7, SD 3.6) years. Changes/year of CD4 count and plasma viremia (VL) were calculated using a linear regression model.

In this longitudinal cohort total Gag, p17- and p24-specific PHPC counts inversely correlated with subsequent VL change/year (r=−0.59,p=0.006; r=−0.53,p=0.015; r=−0.46, p=0.04; respectively, by Pearson's test). Although there was a trend for a positive correlation between total Gag, p17- and p24-specific PHPC counts and change of CD4 counts, it did not reach statistical significance. A mean p17-specific PHPC count of 3.2 (±5.5) was detected in four fast progressors that showed a mean CD4 count decrease/year of 100 (±79) and a mean log₁₀ VL increase/year of 0.2 (±0.2). A mean response of 3,379.8 (±4,334.3) PHPC count was detected in 15 slow progressors that showed a mean of 14 (±22) CD4 count decrease/year and no VL increase (0.0±0.2). At the time of PHPC analysis, the mean p17-specific PHPC counts were significantly different between the two groups (p=0.0092; Welch's t-test) while CD4 count and VL were not p=0.3535 and p=0.12, respectively; two-sample t-test), indicating that measuring PHPC might offer an advantage over conventional assays to forecast the evolution of the disease.

### Example 3

### Individual Characteristics and Samples Analyzed

Individuals receiving antiretroviral therapy (ART) with CD4 counts >300 cells/mm³ (nadir >250 cells/mm³), and HIV RNA PCR <50 copies/ml for >12 weeks Cohort 1: 3 subjects receiving as treatment DermaVir Patches plus continuous ART. Dose: 2 DermaVir Patches (0.05 mg DNA per patch). Cohort 2: 3 subjects receiving as treatment DermaVir Patches+continuous ART. Dose: 4 DermaVir Patches (0.1 mg DNA per patch). Cohort 3: 3 subjects receiving as treatment DermaVir Patches+continuous ART. Dose: 8 DermaVir Patches (0.1 mg DNA per patch).

### Synthetic Peptides, PHPC Assay, and Data Analysis

If not indicated, synthetic peptides, PHPC assay, and data analysis were the same as in Example 1.

### Results

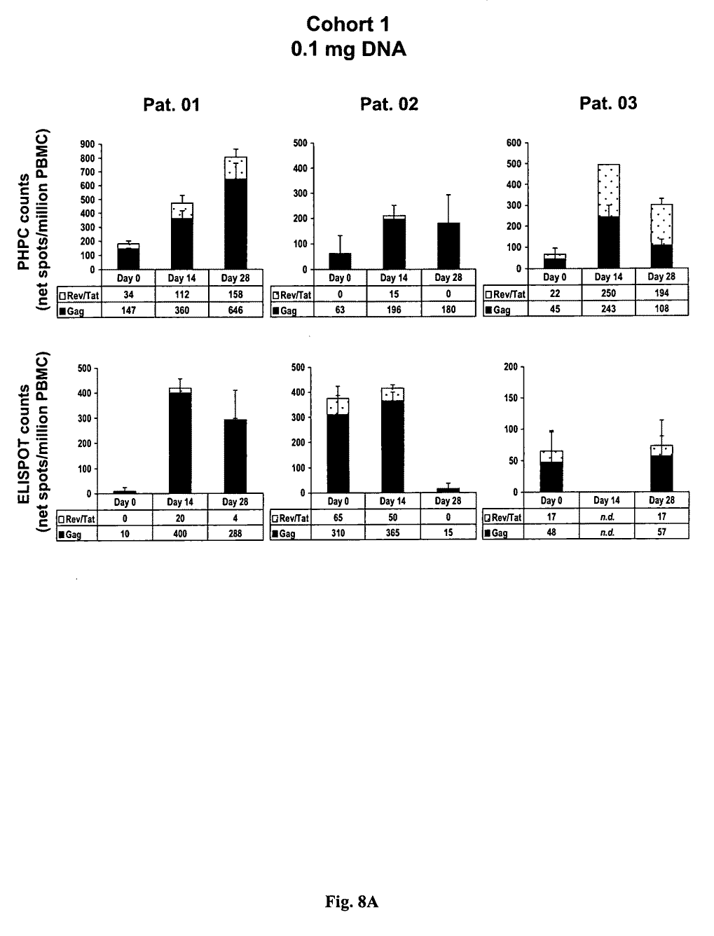

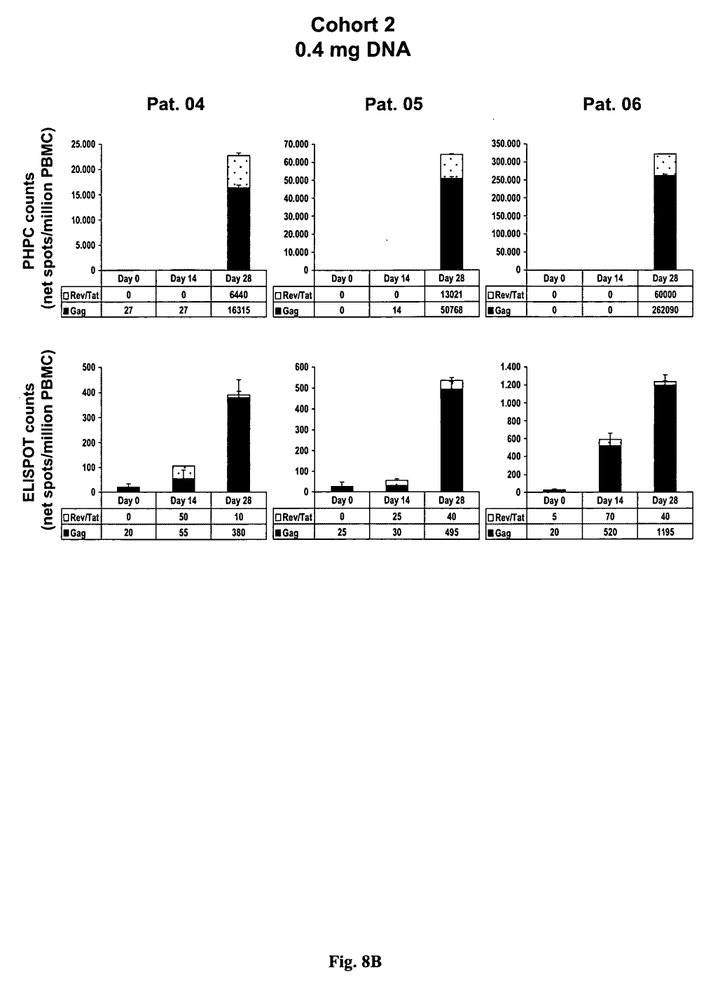

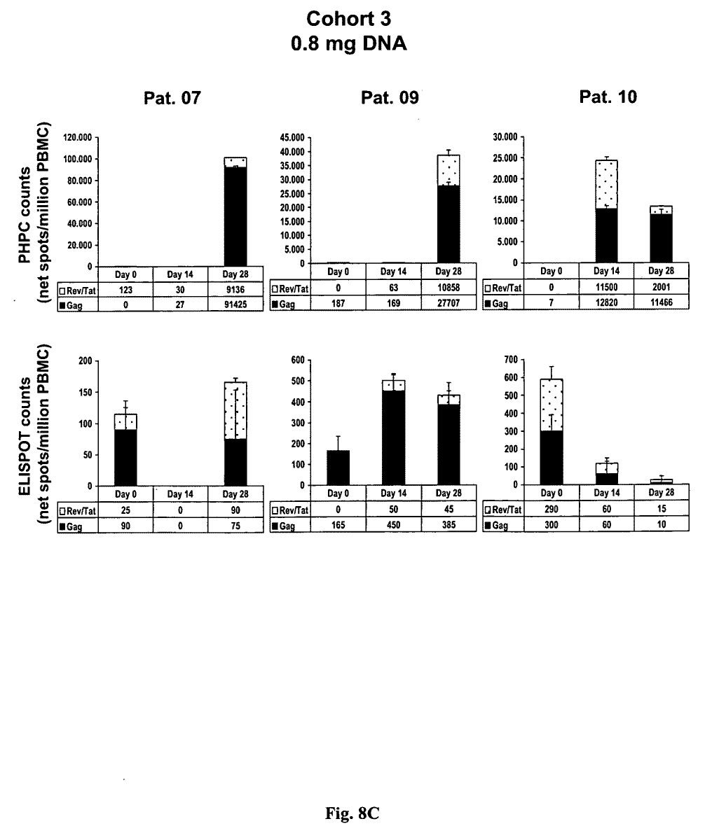

The magnitude of Gag-specific PHPC increased from <200 (baseline) to 2,300-4,000 at week 24 in the low dose, and to 2,500-21,500 and 3,000-37,000 at week 48 in the medium and high dose DermaVir Patch, respectively (FIG. 8, upper panels in each cohort). The broadening of T-cell responses was demonstrated by the induction of new Rev- and Tat-specific PHPC. These were mostly undetectable prior to immunization and increased up to 3,000-30,000 (Rev) and 60-30,000 (Tat) in the medium dose and up to 1,000-7,000 (Rev) and 600-4,000 (Tat) in the high dose, at day 28. In the medium dose cohort, for example, the average frequency of total PHPC and of effector T-cells prior to vaccination were 124 (20); 28 days after vaccination 231,403 (720), and one year later 82,776 (n.d.), indicating >10 fold expansion of effectors and >100 fold of long-lived T-cell precursors. ELISPOT responses were negligible in all cohorts (FIG. 8, lower panels in each cohort).

The magnitude and breadth of HIV-specific T-cell precursors significantly increased in all treated patients in a dose dependent manner, suggesting an excellent immunogenicity profile in human subjects.

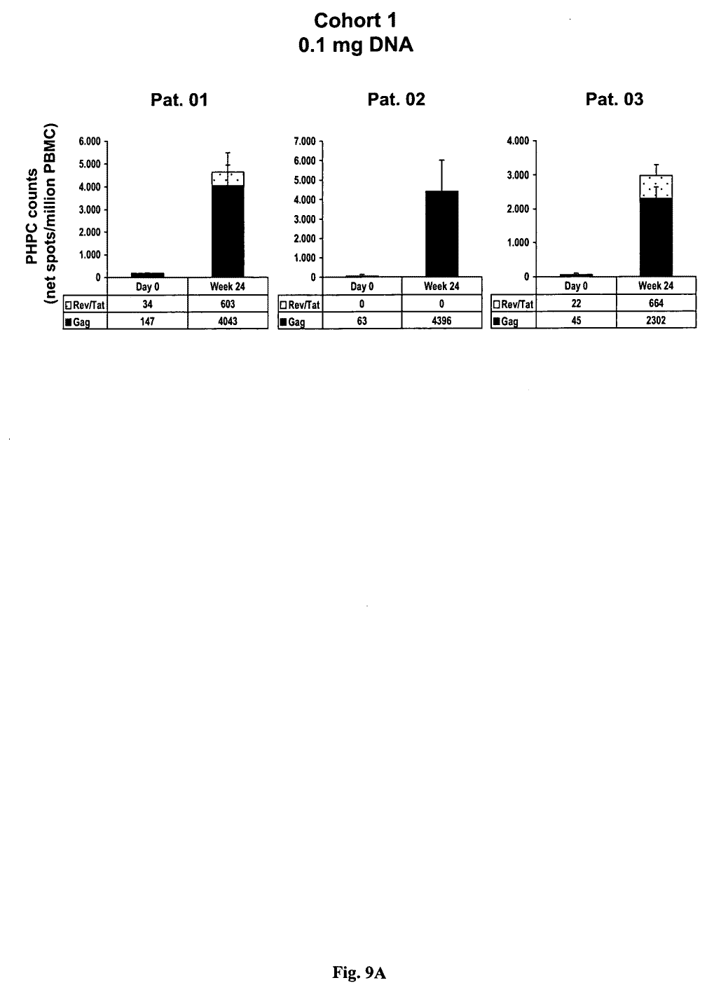

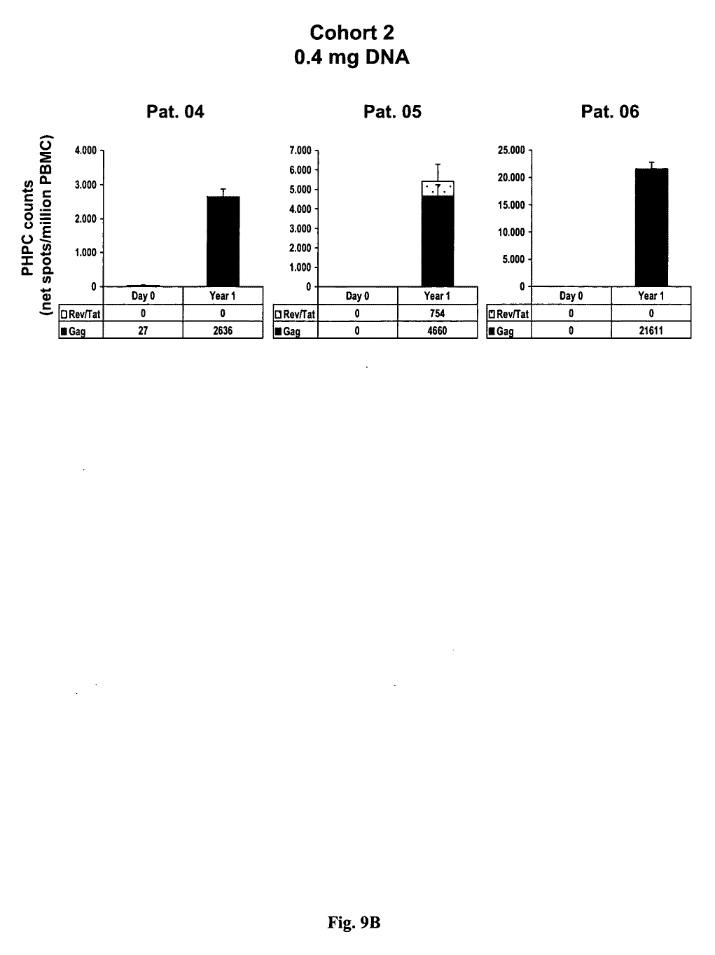

After a single DermaVir Patch treatment long-lived HIV Gag-specific T-cell precursors were detected in all nine HIV-infected subjects, six patients also had long-lived T-cell precursors specific to HIV regulatory proteins Rev and or Tat (FIG. 9).

### Example 4

### Cross-Sectional Analysis: WIHS Cohort Only

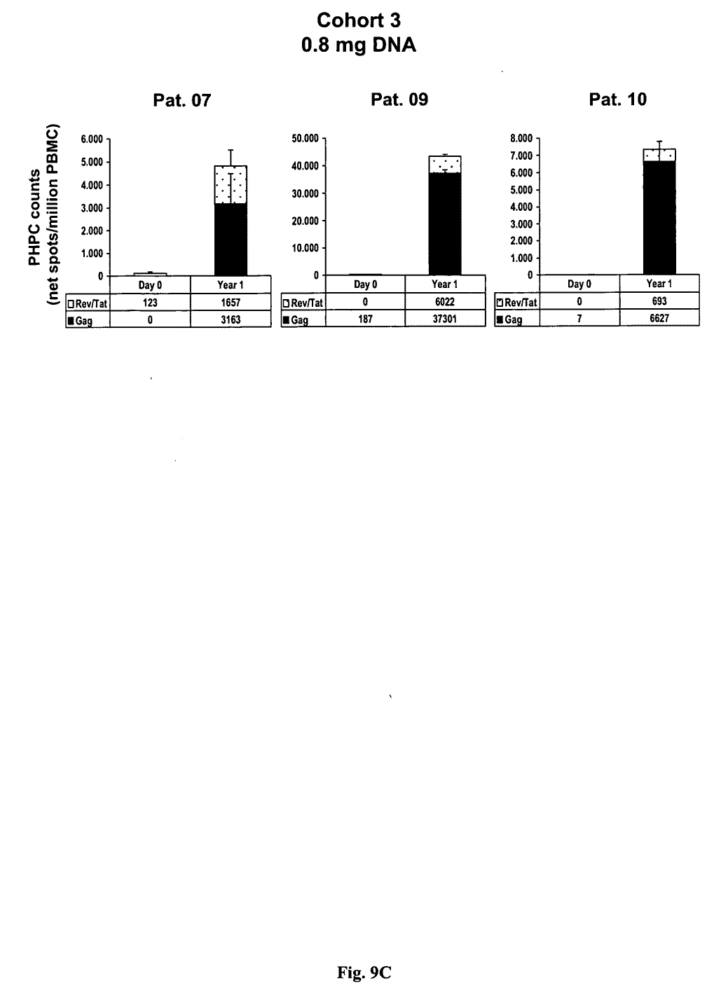

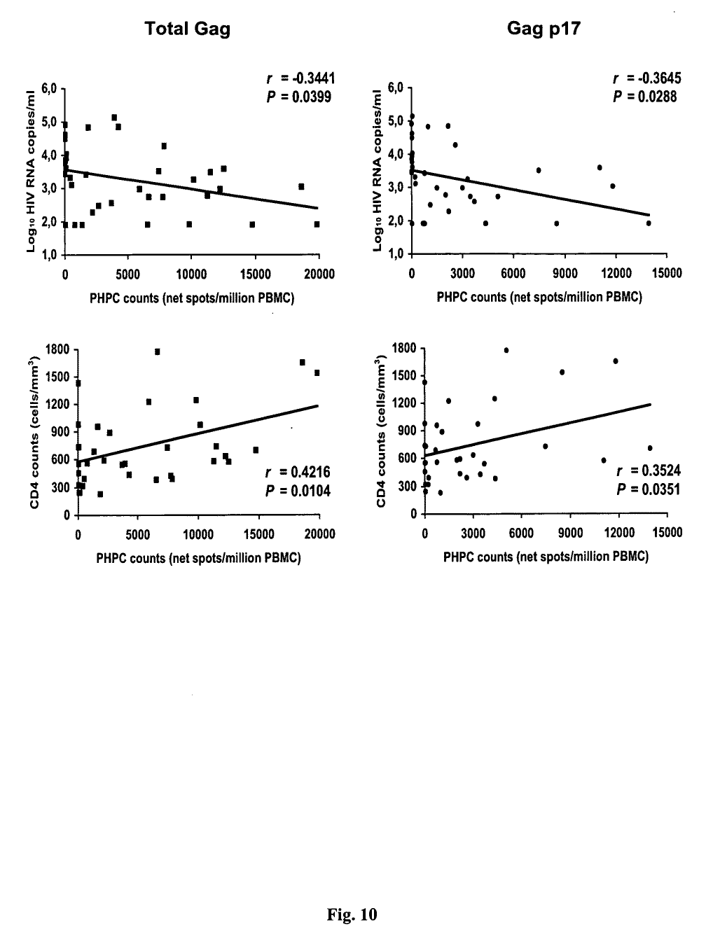

We tested 20 PBMC samples from chronically untreated HIV-1-infected individuals in the WIHS cohort. We further tested 16 additional PBMC samples. Analysis of all samples from the WIHS cohort (n=36) confirmed the correlation between Gag-specific PHPC responses, mainly due to p17 subunit, and either plasma viremia (inverse correlation) or CD4 counts (direct correlation) (FIG. 10)

### Example 5

Cross-Sectional Analysis: All 68 Samples (32 from the Italian/Canadian Cohort and 36 from the WIHS Cohort)

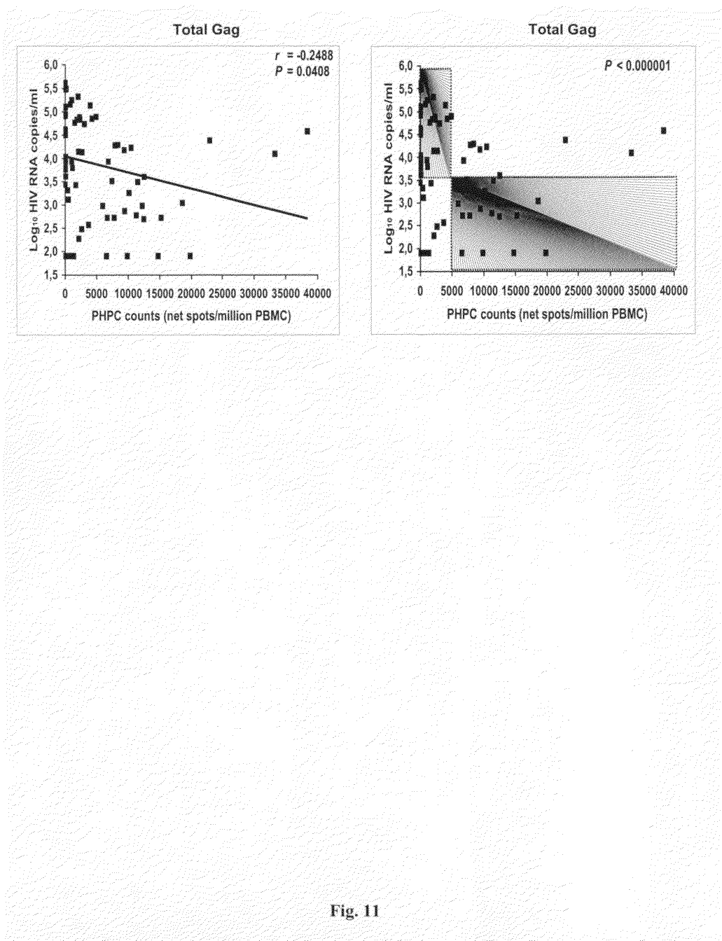

Based on sample distribution of the cross-sectional data, we assigned a range of HIV-specific PHPC response that can be correlated with a certain range of viral load. Taken together the cross-sectional data of untreated, chronically HIV-1-infected individuals (n=68 samples), the PHPC cut-off value was determined by ROC curve analysis (based on the assumption that a positive PHPC response will be associated with log₁₀ VL <3.5 copies/ml). The Gag cut-off value was 4,895 PHPC counts, that is Gag-specific PHPC response higher than 4,895 counts are likely to be associated with VL lower than 3.5 log10 copies/ml. Using this cut-off value, we found a statistically significant difference between individuals with high log₁₀ VL/low PHPC counts versus low log₁₀ VL/high PHPC counts (FIG. 11)

### Example 6

### Longitudinal Analysis: WIHS Cohort Only

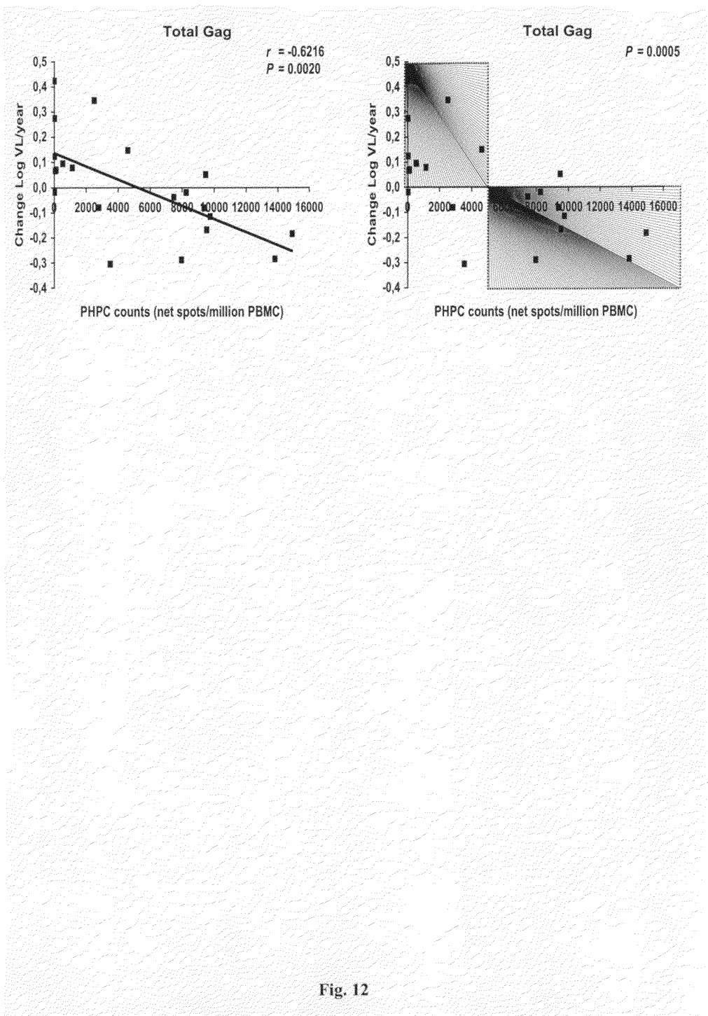

Based on the longitudinal analysis of 22 individuals from the WIHS cohort, we found that a Gag-specific PHPC response ≧4,895 counts was associated with stable plasma VL over time and a statistically significant difference between individuals with increase log₁₀ VL/low PHPC counts versus decrease log₁₀ VL/high PHPC counts over time (FIG. 12).

What is claimed is:

1. A diagnostic assay for antigen-specific T-cell responses, the steps comprising:
a. Stimulating PBMC with one or more specific antigens
b. Culturing PBMC to expand T-cells
c. Restimulating expanded T-cells with one or more specific antigens
d. Measuring cytokine release.

2. The assay of claim 1, wherein the antigens are derived from a virus.

3. The assay of claim 2, wherein the virus is HIV-1.

4. The assay of claim 1, wherein the antigens are multiple peptide pools.

5. The assay of claim 1, wherein the antigens are one peptide pools.

6. The assay of claim 1, wherein the antigens are one peptide.

7. The assay of claim 3, wherein the multiple peptide pools are selected from the group consisting of Gag, Nef, Tat and Rev proteins.

8. The assay of claim 7, wherein the peptide pool represents the HIV-1 Gag protein subgroup p17 and/or p24.

9. The assay of claim 1, wherein the PBMC are stimulated for at least 2 days.

10. The assay of claim 9, wherein the PBMC are stimulated for 7-21 days.

11. The assay of claim 2, wherein the PBMC are stimulated for 7-14 days.
